(19) United States
(12) Patent Application Publication
(10) Pub. No.: US 2021/0173368 A1
(43) Pub. Date: Jun. 10, 2021

(54) Smart Hotel System

(71) Applicant: Board of Trustees of Michigan State University, East Lansing, MI (US)

(72) Inventors: Wolfgang BAUER, East Lansing, MI (US); Natalie MCQUADE, Ann Arbor, MI (US); Ryan MULKA, Milford, MI (US); Mitchell LOE, Williamston, MI (US); David OSINSKI, Sterling Heights, MI (US); Michele WEBER, Fowlerville, MI (US); Jonathan SPIGHT, North Barrington, IL (US)

(73) Assignee: Board of Trustees of Michigan State University, East Lansing, MI (US)

(21) Appl. No.: 17/111,664

(22) Filed: Dec. 4, 2020

Related U.S. Application Data
(60) Provisional application No. 62/944,430, filed on Dec. 6, 2019.

(51) Int. Cl. G05B 19/042; G07C 9/29; G07C 9/00; G06Q 10/02; G06Q 50/12; G06Q 10/10; G06Q 10/00; A61L 2/24

(52) U.S. Cl. CPC G05B 19/042; G07C 9/29; G07C 9/00904; G06Q 10/02; G06Q 50/12; G06Q 10/109; A61L 2202/25; A61L 2/24; G05B 2219/25011; A61L 2202/17; A61L 2202/14; A61L 2202/16; G06Q 10/20

(57) ABSTRACT

In accordance with the present invention, a smart hotel system is provided. In one aspect, the system includes an occupancy sensor and a processor. Another aspect provides an occupancy sensor associated with at least one hotel room and a programmable controller or processor configured to execute instructions stored in a nontransitory computer-readable medium. A further aspect includes programmed software instructions which include obtaining data indicative of occupancy status of the hotel room from the occupancy sensor and displaying the occupancy status of the hotel room. In another aspect, a controller or processor obtains data indicative of a cleaning status of at least one hotel room and displays the cleaning status of the hotel room.

### CROSS-REFERENCE TO RELATED APPLICATIONS

This application claims the benefit of U.S. Provisional Application No. 62/944,430, filed on Dec. 6, 2019. The entire disclosure of the above application is incorporated herein by reference.

### FIELD

The present disclosure relates to a smart hotel system and more particularly to a system including a hotel room occupancy sensor.

### BACKGROUND AND SUMMARY

There are approximately 17 million hotel rooms in more than 180,000 hotels around the world. Every day these hotel rooms are serviced by hundreds of thousands of service personnel, who have to knock on every door, multiple times, to announce “housekeeping.” If the room is occupied, they have to return at a later time to try again, often repeatedly.

In accordance with the present invention, a smart hotel system is provided. In one aspect, the system includes an occupancy sensor and a processor. Another aspect provides an occupancy sensor associated with at least one hotel room and a programmable controller or processor configured to execute instructions stored in a nontransitory computer-readable medium. A further aspect includes programmed software instructions which include obtaining data indicative of occupancy status of the hotel room from the occupancy sensor and displaying the occupancy status of the hotel room. In another aspect, a controller or processor obtains data indicative of a cleaning status of at least one hotel room and displays the cleaning status of the hotel room.

The system according to the present disclosure is advantageous over traditional methods of cleaning hotel rooms. For example, the system allows service personnel to focus their attention on currently unoccupied rooms in need of cleaning, and it enables inspection personnel to conveniently examine newly cleaned rooms. All rooms can thus be serviced in the minimum time on the first attempt and without disturbing any of the hotel guests. Additional advantages and features of the present system can be ascertained from the following description and claims taken in conjunction with the appended drawings.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a functional block diagram showing an exemplary smart hotel system according to the present disclosure;

FIG. 2 is a diagrammatic display showing an exemplary page on a mobile device of the smart hotel system;

FIG. 3 is a plan view showing an exemplary occupancy sensor; and

FIG. 4 is a flowchart showing control logic employed in the smart hotel system.

### DETAILED DESCRIPTION

Example embodiments will now be described more fully with reference to the accompanying drawings.

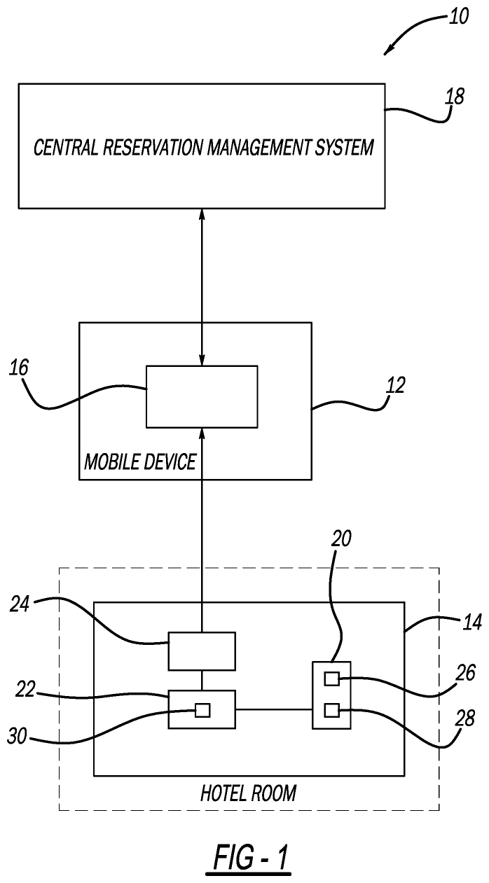

As shown in FIG. 1, a smart hotel system 10 is provided. System 10 provides the current cleaning status of hotel rooms on any given hotel floor, current occupancy status of hotel rooms and other details about the hotel rooms such as outlets and light fixtures that are conditionally enabled when a guest is present. Smart hotel system 10 includes one or more mobile devices 12 and one or more occupancy sensors 14. Each mobile device 12 (e.g., a tablet, a smartphone, a laptop, or other similar device) is mounted on a hotel service cart or carried by service personnel. Each mobile device 12 includes a processor that is configured to execute instructions stored in a nontransitory computer-readable medium, such as a read-only memory (ROM) and/or random-access memory (RAM).

Each mobile device 12 includes a software application 16. The functions of the software application 16 is accessed using, for example, native application editions of the software and/or web applications of the software.

One or more mobile devices 12 and one or more occupancy sensors 14 are configured to, using the software application 16, communicate via wireless communication protocol. The wireless communication protocol includes an internet, Wi-Fi, Bluetooth®, or cellular connection or any other wireless communication protocol, for example. For example, the one or more mobile devices 12 and the one or more occupancy sensors 14 communicate with each other over the internet via a Wi-Fi connection to a Wi-Fi router located in or associated with the hotel.

One or more mobile devices 12 are also in communication with a central reservation management system 18 via, for example, an internet, Wi-Fi, Bluetooth®, or cellular connection or any other wireless communication protocol, for example. For example, each mobile device 12 communicates with the central reservation management system 18 over the internet via a Wi-Fi connection to a Wi-Fi router located in or associated with the hotel. Central reservation management system 18 includes data such as the cleaning status of each hotel room, the occupancy status of each hotel room, and any other relevant information about each hotel room (e.g., whether or not any special cleaning requests are noted for a particular room, whether or not late checkout has been requested for a particular room, or whether or not a particular room only needs a cursory cleaning based on the guests staying multiple nights in the room). Central reservation management system 18 communicates this data to the software application 16 included on each mobile device 12. In this way, a user of the mobile device 12 may, using the software application 16, view the check-out status of any hotel room, view current cleaning status of any hotel room, update the cleaning status of any hotel room, create workers orders for hotel rooms, and request emergency assistance to any hotel rooms, for example.

Each occupancy sensor 14 is associated within a particular hotel room and communicates to a user of each mobile device 12 whether someone is currently in the hotel room. Each occupancy sensor 14 includes a cardholder device 20, a printed circuit board (PCB) 22 and a controller 24. Cardholder device 20 is in communication with the PCB 22 and includes a cardholder 26 and a RFID antenna 28. Cardholder device 20 detects when a keycard has been inserted into the cardholder 26 and activates LEDs 30 electrically coupled to the PCB 22 accordingly. For example, if a valid keycard has been inserted into the cardholder 26 then a green LED is activated to notify the occupant of the room that the keycard is valid. If an invalid key card has been inserted into the cardholder 26 then a red LED is activated to notify the occupant of the room that the keycard is invalid. In response to a valid keycard being inserted into the cardholder 26, the system 10 will conditionally power one or more outlets and/or light fixtures associated with the room. LEDs 30 associated such outlets and/or light fixtures will activate to notify the occupant of the room that such outlets and/or light fixtures are now operable (powered). It should be understood that in the event that an invalid keycard is inserted into the cardholder 26, LEDs 30 associated with such outlets and/or light fixtures will not activate, thereby notifying the occupant that such outlets and/or light fixtures are not operable (powered). It is understood that the keycard used for the cardholder device 20 is the same keycard used to access entry into the hotel room. In this way, the keycard must be removed when leaving the hotel room in order to gain re-entry into the hotel room.

It should also be understood that, in some configurations, the occupancy sensor 14 can be RFID receivers for properly equipped room keys, or key slots for metal hotel keys. These slots can have the dual function of allowing power to flow to certain power outlets and light fixtures only if the room is occupied, which is also an energy conservation measure. Use of other occupancy sensors, such as infrared sensors or motion detectors, are also possible.

Controller 24 is electrically coupled (e.g., via wires) to the PCB 22 and is in communication with the software application 16. Controller 24 communicates to the software application 16 whether or not the hotel room is occupied based on the occupancy sensor 14. In this way, the user of the mobile device 12 focuses on rooms that need cleaning and are unoccupied.

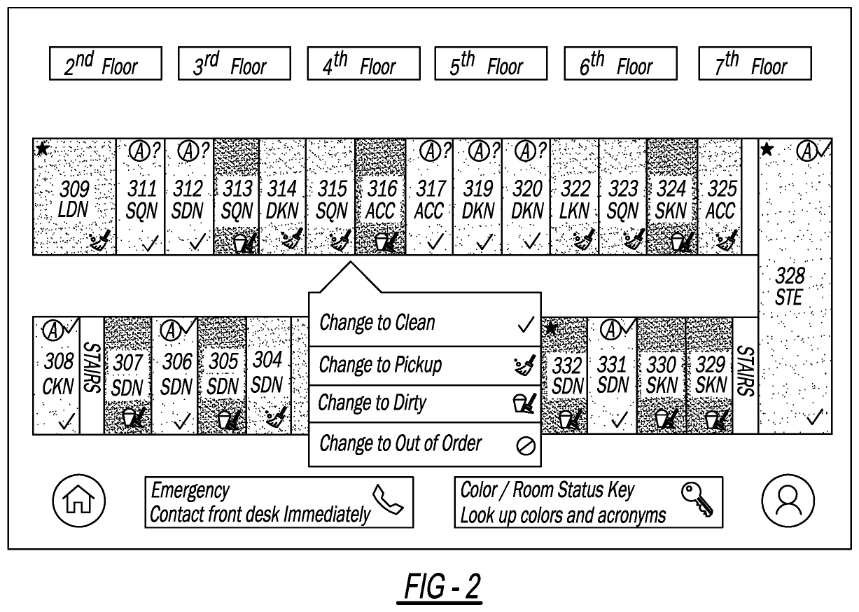

FIG. 2 shows an example page of the software application 16 user interface on the mobile device 12. In such example, the layout of the third floor of a hotel is shown including the rooms on third floor and the current cleaning/occupancy status of each room. That is, the current cleaning/occupancy status of each room may be shown using color, symbols and/or text.

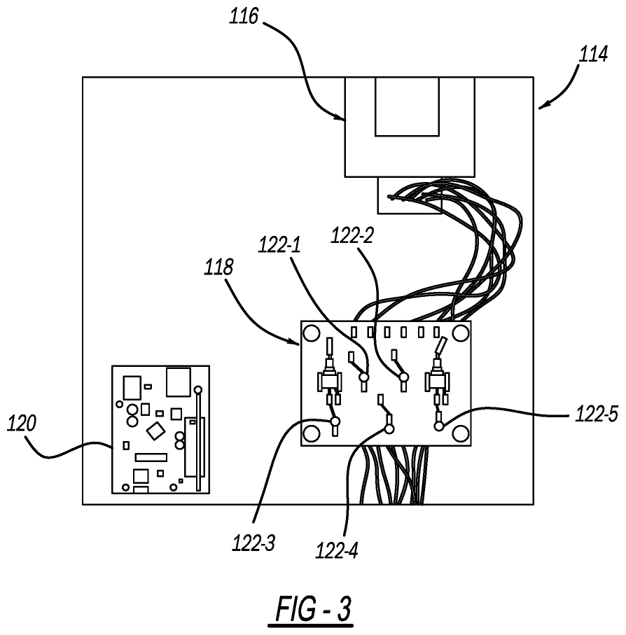

FIG. 3 shows an occupancy sensor 114 including a cardholder device 116, a printed circuit board (PCB) 118 and a controller 120. PCB 118 is wired to the cardholder device 116 and to the controller 120. The PCB also includes LEDS 122-1, 122-2, 122-3, 122-4, 122-5 electrically coupled thereto.

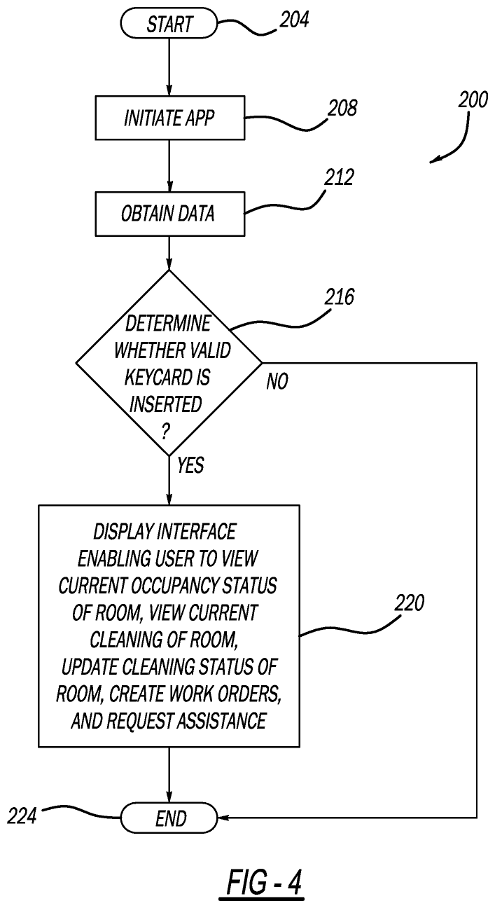

With reference to FIG. 4, a flowchart illustrating exemplary software control logic 200 is shown. The control logic 200 begins at 204 when, for example, one of the mobile devices 12 is turned on. At 208, the control logic 200 initiates, using a processor of the mobile device 12, the software application 16. As an example, the software application 16 may be initiated by opening a local copy of the application stored in the nontransitory computer-readable medium of the mobile device 12.

At 212, the control logic 200 obtains, using the software application 16, data indicative of cleaning/occupancy status of one or more hotel rooms of the hotel. For example, the data may include information from the central reservation management system 18 such as the check-out status of any hotel room and/or the current cleaning status of any hotel room. At 216, the control logic 200 determines, using the software application 16, whether a valid keycard is inserted into the occupancy sensor 14. That is, the occupancy sensor 14 may communicate with the software application 16 on whether a valid keycard has been inserted in the cardholder 26, which signifies that the room is currently occupied. If so, the control logic 200 proceeds to 220; otherwise, the control logic 200 proceeds to 224 and ends.

At 220, the control logic 200 may display, using the software application 16, an interface enabling the user to, for example, view the current occupancy status of hotel rooms, view current cleaning status of hotel rooms, update the cleaning status of hotel rooms, create workers orders for hotel rooms, and request emergency assistance to hotel rooms. The control logic 200 then proceeds to 224 and ends. System 10 of the present disclosure provides the benefit of determining when guests are present in each hotel room, thereby no longer disturbing guests while providing daily cleaning services. The system 10 of the present disclosure also provides the benefit of enabling inspection personnel to examine newly cleaned rooms.

While various embodiments have been disclosed, other variations are envisioned. For example, controller 24 may be attached on the PCB 22 instead of connected thereto via wires. Furthermore, additional or different electronic components may be employed other than those specifically described. Variations are not to be regarded as a departure from the present disclosure, and all such modifications are intended to be included within the scope and spirit of the present invention.

What is claimed is:

1. A system comprising:
(a) a first occupancy sensor associated with a first hotel room; and
(b) a first controller configured to execute programmed instructions stored in a nontransitory computer-readable medium, wherein the instructions include:
(i) obtaining data indicative of occupancy status of the first hotel room from the first occupancy sensor; and
(ii) transmitting the occupancy status of the first hotel room to a remote mobile device.

2. The system of claim 1, wherein the first occupancy sensor includes:
a cardholder located within the first hotel room and configured to receive a key card,
wherein the data indicative of occupancy status includes whether or not a valid keycard is received in the cardholder.

3. The system of claim 2, wherein the first occupancy sensor further includes:
a printed circuit board (PCB) electrically coupled to the cardholder; and
a first light emitting diode (LED) electrically coupled to the PCB,
wherein when a valid keycard is received in the cardholder, the first LED is activated with a first color to notify an occupant of the first hotel room that the keycard is valid, and when an invalid keycard is received in the cardholder, the first LED is activated with a second color to notify the occupant that the keycard is invalid.

4. The system of claim 3, wherein the first occupancy sensor further includes:
a second light emitting diode (LED) electrically coupled to the PCB and associated with one of an outlet and light fixture in the first hotel room,
wherein when the valid keycard is received in the cardholder, the second LED is activated with a third color to notify the occupant that the one of the outlet and light fixture is operable, and when the invalid keycard is received in the cardholder, the second LED is activated with a fourth color to notify the occupant that the one of the outlet and light fixture is inoperable.

5. The system of claim 1, wherein the remote mobile device is configured to display the occupancy status of the first hotel room.

6. The system of claim 1, wherein the remote mobile device is a smartphone or tablet.

7. The system of claim 1, wherein the remote mobile device is configured to display the occupancy status of the first hotel room.

8. The system of claim 7, further comprising a central reservation management system in communication with the remote mobile device and configured to provide data to the remote mobile device including cleaning status of the first hotel room.

9. The system of claim 1, further comprising:
(c) a second occupancy sensor associated with a second hotel room; and
(d) a second controller configured to execute programmed instructions stored in a nontransitory computer-readable medium, wherein the instructions include:
(i) obtaining data indicative of occupancy status of the second hotel room from the second occupancy sensor; and
(ii) transmitting the occupancy status of the second hotel room to the remote mobile device.

10. The system of claim 9, wherein the remote mobile device is configured to display the occupancy status of the first and second hotel rooms.

11. The system of claim 1, further comprising a central reservation management system in communication with the remote mobile device and configured to provide data to the mobile device including cleaning status of the first and second hotel rooms.

12. The system of claim 1, wherein the first controller is configured to automatically transmit anti-viral cleaning instructions to the remote mobile device in response to the data from the first occupancy sensor.

13. A system comprising:
(a) an occupancy sensor associated with a hotel room;
(b) a remote mobile device;
(c) a controller configured to execute programmed instructions stored in a nontransitory computer-readable medium, wherein the instructions include:
(i) obtaining data indicative of occupancy status of the hotel room from the occupancy sensor; and
(ii) transmitting the occupancy status of the hotel room to the remote mobile device; and
(d) a central reservation management system in communication with the remote mobile device and configured to provide data to the remote mobile device including cleaning status of the hotel room,
the remote mobile device is configured to display an interface enabling a user to view the occupancy status of the hotel room, view and update cleaning status of the hotel room, and create work orders for the hotel room,
the first occupancy sensor further includes:
a cardholder located within the hotel room and configured to receive a key card, the data indicative of occupancy status includes whether or not a valid keycard is received in the cardholder;
a printed circuit board (PCB) electrically coupled to the cardholder;
a first light emitting diode (LED) electrically coupled to the PCB;
a second light emitting diode (LED) electrically coupled to the PCB and associated with one of an outlet and light fixture in the first hotel room;
wherein when a valid keycard is received in the cardholder, the first LED is activated with a first color to notify an occupant of the first hotel room that the keycard is valid, and when an invalid keycard is received in the cardholder, the first LED is activated with a second color to notify the occupant that the keycard is invalid; and
wherein when the valid keycard is received in the cardholder, the second LED is activated with a third color to notify the occupant that the one of the outlet and light fixture is operable, and when the invalid keycard is received in the cardholder, the second LED is activated with a fourth color to notify the occupant that the one of the outlet and light fixture is inoperable.

14. A method comprising:
(a) obtaining data indicative of occupancy status of multiple hotel rooms from occupancy sensors;
(b) automatically determining the occupancy of the hotel rooms using programmable controllers, based at least in part on the data; and
(c) displaying the determined occupancy of the hotel rooms on a mobile device in response to the determination to assist in scheduling cleaning of at least one of the hotel rooms.

15. The method of claim 14, wherein each occupancy sensor includes:
a cardholder located within a respective hotel room and receiving a key card,
automatically determining from the data indicative of occupancy status whether or not a valid keycard is received in the cardholder.

16. The method of claim 15, wherein each occupancy sensor further includes:
a printed circuit board (PCB) receiving data from the cardholder; and
electrically coupling a first light emitting diode (LED) to the PCB,
automatically activating the first LED with a first color when a valid keycard is received in the cardholder to notify an occupant of the respective hotel room that the keycard is valid, and automatically activating the first LED with a second color when an invalid keycard is received in the cardholder to notify the occupant of the respective hotel room that the keycard is invalid.

17. The method of claim 16, wherein each occupancy sensor further includes:
electrically coupling a second light emitting diode (LED) to the PCB, the second LED associated with one of an outlet and light fixture in the respective hotel room,
automatically activating the second LED with a third color when the valid keycard is received in the cardholder to notify the occupant that the one of the outlet and light fixture is operable, and automatically activating the second LED with a fourth color when the invalid keycard is received in the cardholder to notify the occupant that the one of the outlet and light fixture is inoperable.

18. The method of claim 14, further comprising:
obtaining data indicative of cleaning status of multiple hotel rooms from a central reservation management system; and
displaying the cleaning status of the hotel rooms on the mobile device.

19. The method of claim 18, wherein the mobile device is a smartphone or tablet.

20. The method of claim 14, further comprising automatically transmitting anti-viral cleaning instructions to the mobile device in response to the determination to assist in scheduling cleaning of at least one of the hotel rooms.

21. The method of claim 14, further comprising enabling a user to view and update cleaning status of the hotel room and create work orders for the hotel room from the mobile device.
